(19) United States
(12) Patent Application Publication
(10) Pub. No.: US 2025/0195395 A1
(43) Pub. Date: Jun. 19, 2025

(54) COSMETIC ADHESIVE COMPOSITIONS

(71) Applicant: L'OREAL, PARIS (FR)

(72) Inventors: David CHAN, ORADELL, NJ (US); Allison PERNER, METUCHEN, NJ (US)

(73) Assignee: L'OREAL, PARIS (FR)

(21) Appl. No.: 18/539,466

(22) Filed: Dec. 14, 2023

(51) Int. Cl. A61K 8/87; A61K 8/81; A61Q 1/10

(52) U.S. Cl. CPC A61K 8/87; A61K 8/8147; A61Q 1/10; A61K 2800/34; A61K 2800/594; A61K 2800/94

(57) ABSTRACT

A cosmetic adhesive composition is provided. The cosmetic adhesive composition includes water; from about 10% to 20% by weight of one or more acrylic latex polymers having a glass transition temperature (T_{g}) less than about −40° C.; one or more aqueous polyurethane dispersions; and one or more saccharide esters of at least one C2-C4 organic acid. The one or more acrylic latex polymers having a glass transition temperature less than about −40° C. and the one or more aqueous polyurethane dispersions are present in a ratio of concentration by weight that is in a range from about 1:3 to about 3:1. Methods of adhering false eyelashes and false brows are also provided.

### TECHNICAL FIELD

The present disclosure is drawn to cosmetic adhesive compositions, such as those suitable for adhering false lashes or false brows for extended periods of time.

### BACKGROUND

To achieve a desired aesthetic for a user's eyelashes and brows, false lashes and false brows may be utilized. These may be adhered using an adhesive or “glue.” The inventors of the present invention have found that it is not only important for such glue to provide not only adhesion to body surfaces, but also to provide sufficient cohesion of the glue itself, in order to make the glue last sufficiently long.

### BRIEF SUMMARY

A cosmetic adhesive is provided. The cosmetic adhesive composition may include water; about 10% to 20% by weight of one or more acrylic latex polymers having a glass transition temperature (T_{g}) less than about −40° C.; one or more aqueous polyurethane dispersions; and one or more saccharide esters of at least one C2-C4 organic acid. The one or more acrylic latex polymers having a low glass transition temperature and the one or more aqueous polyurethane dispersions are present in a ratio of concentration by weight of the one or more acrylic latex polymers having a low glass transition temperature to concentration by weight of the one or more aqueous polyurethane dispersions that is in a range from about 1:3 to about 3:1.

Desirably, the ratio of concentration by weight of the one or more acrylic latex polymers having a low glass transition temperature to concentration by weight of the one or more aqueous polyurethane dispersions the in a range from about 1:1 to about 2:1.

Desirably, the one or more acrylic latex polymers having a glass transition temperature (T_{g}) may have a T_{g} less than about −50° C.

Desirably, the one or more aqueous polyurethane dispersions is formed by reacting a polyester prepolymer with a non-phenylated isocyanate.

Desirably, the composition is substantially free of one or more of waxes, oils silicones and/or C2-C6 polyols.

Desirably, the composition optionally includes one or more additional adhesive polymers, and wherein the total concentration by weight of the one or more acrylic latex polymers having a glass transition temperature (T_{g}) less than about −40° C.; the one or more aqueous polyurethane dispersions; the one or more saccharide esters of at least one C2-C4 organic acid, and the one or more additional adhesive polymers is in a range from about 30% to about 70%.

Desirably, the one or more acrylic latex polymers has a glass transition temperature (T_{g}) less than about −40° C. is present in the composition in a concentration by weight from about 10% to about 20% of the composition.

Desirably, the one or more aqueous polyurethane dispersions is present in the composition in a concentration by weight from about 8% to about 20% of the composition.

Desirably, the one or more saccharide esters of at least one C2-C4 organic acid is present in an amount of 5% to about 30% by weight of the composition. The composition may include one, a plurality or even all of the above-described particularities.

According to another aspect, a method of adhering a false eyelash, includes applying the composition described above to a surface selected from a false lash, a natural lash, or combinations thereof to form an active adhesive surface. It further includes contacting the active adhesive surface with a corresponding mating surface (false lash or natural lash) to form an augmented lash. For example the adhesive may be applied to false lash and then attached to a corresponding natural lash; or the adhesive may be applied to a natural lash and then attached to a corresponding false lash. Similarly, the method may further include adhering a false eyebrows, and may include applying the composition described above to a surface selected from a false brow, a natural brow, or combinations thereof to form an active adhesive surface. It further includes contacting the active adhesive surface with a corresponding mating surface (false brow or natural brow) to form an augmented brow. For example, the adhesive may be applied to false brow and then attached to a corresponding natural brow; or the adhesive may be applied to a natural brow and then attached to a corresponding false brow. In other embodiments the cosmetic adhesive is applied to skin surface, such as skin on the eyelid or skin of the brow area.

### DETAILED DESCRIPTION

Other than in the operating examples, or where otherwise indicated, all numbers expressing quantities (e.g., concentrations, ratios, and the like) of ingredients and/or reaction conditions are to be understood as being modified in certain embodiments by the term “about,” meaning within 10% to 15% of the indicated number (e.g. “about 10%” means 8.5% to 11.5% such as 9% to 11%, and “about 2%” means from 1.7% to 2.3 such as from 1.8% to 2.2%).

Similarly, for ratios, the modifier “about” means within 10% or 15% of the number. For example, about 4:1 means from 3.4:1 to 4.6:1, preferably 3.6:1 to 4.4:1. As readily understood by one skilled in the art, where the first ingredient in a ratio is less than the second, then a ratio may be expressed “inversely.” For example, if a second ingredient, B is present in an amount or concentration that is 2.5 times greater than that of ingredient A, this may be identified as an A:B ratio of 1:2.5. “About 1:10,” means from 1:8.5 to 1:11.5, preferably 1:9 to 1:11. Unless otherwise indicated, all concentrations shown as percentages are concentrations by weight and also, unless otherwise indicated, relate to the entire cosmetic adhesive composition as a whole.

As used herein, the term “at least one” means one or more.

As used herein, the term “free” or “completely free (of a component)” refers to compositions that do not contain the component in any measurable degree by standard means. As used herein, the term “substantially free (of a component)” refers to compositions that contain no appreciable amount of the component, for example, no more than about 1% by weight, or no more than about 0.5% by weight, or no more than about 0.3% by weight, such as no more than about 0.1% by weight, based on the weight of the composition.

In some embodiments, the composition may be, for example, a single phase.

### Water

The adhesive composition includes water. In some embodiments, water may be present in amounts of up to 65% by weight of the composition. In some embodiments, water may be present in amounts from 15%, 20%, 25%, 30%, or 35% up to 45%, 50%, 55%, 60%, or 65% by weight of the composition, including any combinations or subrange thereof.

Compositions of the present invention include adhesive polymers such as those described below: one or more acrylic latex polymer having a low glass transition temperature, one or more aqueous polyurethane dispersion, one or more saccharide esters of at least one C2-C4 organic acid, as well as optional other adhesive polymers. The total amount the adhesive polymers in the composition may range from about 30%, 35% 40% or 45% to about 45%, 50%, 55%, 60%, 65%, or 70% by weight.

### Acrylic Latex Polymer Having a Low Glass-Transition Temperature (T_{g})

In some embodiments, the cosmetic adhesive composition may include a acrylic latex polymer having a low glass transition temperature (T_{g}). Such acrylic latex polymers will have a T_{g} value no greater than −40° C., and preferably no greater than −45° C., and preferably no greater than −50° C., −55° C., or −60° C.

In some embodiments, the acrylic latex polymer may be an acrylic latex polymer lacking styrene groups. In some embodiments, the acrylic latex polymer may be Acrylates/ethylhexyl acrylates copolymer, which may be commercially available from Kobo Products, Inc. and Daito Kasei Kogyo Co., Ltd., under the trade names Daitosol 5500GM, and/or Daitosol 5500GX. This product may be sold in the form of an emulsion that contains water, ethyl methacrylates/N-butyl acrylates/2-methylhexyl acrylates copolymer (aka acrylates/ethylhexyl acrylates copolymer), and Laureth-20. Daitosol 5500GM and Daitosol 5500GX are disclosed to have a glass transition temperature of −65° C.

In some embodiments, the acrylic latex polymer may be present in amounts from 5%, 10%, or 15% to about 15%, 20%, 25%, 30%, or 35% by weight of the composition, including any combinations or subrange thereof.

### Polyurethane Adhesive

According to the present invention, compositions comprising at least one aqueous polyurethane dispersion are provided. The “aqueous polyurethane dispersion” may be the aqueous polyurethane dispersions disclosed in U.S. Pat. Nos. 7,445,770 and/or 7,452,770, the entire contents of both of which are hereby incorporated by reference.

More specifically, the aqueous polyurethane dispersions of the present invention are preferably the reaction products of:
A) a prepolymer according to the formula:

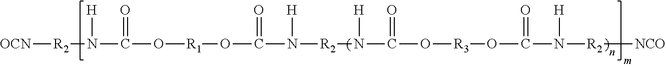

wherein R₁ represents a bivalent radical of a dihydroxyl functional compound, R₂ represents a hydrocarbon radical of an aliphatic or cycloaliphatic polyisocyanate, R₃ represents a radical of a low molecular weight diol, optionally substituted with ionic groups, n is from 0 to 5, and m is >1;
B) at least one chain extender according to the formula: H₂N—R₄—NH₂ wherein R₄ represents an alkylene or alkylene oxide radical not substituted with ionic or potentially ionic groups; and
C) at least one chain extender according to the formula: H₂N—R₅—NH₂ wherein R₅ represents an alkylene radical substituted with ionic or potentially ionic groups.

Suitable dihydroxyl compounds for providing the bivalent radical R₁ include those having two hydroxy groups and having number average molecular weights of from about 700 to about 16,000, and preferably from about 750 to about 5000. Examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. The polyester polyols, polyether polyols and polyhydroxy polycarbonates are preferred. Mixtures of various such compounds are also within the scope of the present invention.

Suitable polyisocyanates for providing the hydrocarbon radical R₂ include organic diisocyanates having a molecular weight of from about 112 to 1,000, and preferably from about 140 to 400. Preferred diisocyanates are those represented by the general formula R₂(NCO)₂ indicated above in which R₂ represents a divalent aliphatic hydrocarbon group having from 4 to 18 carbon atoms, a divalent cycloaliphatic hydrocarbon group having from 5 to 15 carbon atoms, a divalent araliphatic hydrocarbon group having from 7 to 15 carbon atoms or a divalent aromatic hydrocarbon group having 6-15 carbon atoms. Examples of the organic diisocyanates which are suitable include tetramethylene diisocyanate, 1,6-hexamethylene diisocyanate, dodecamethylene diisocyanate, cyclohexane-1,3-and-1,4-diisocyanate, 1-isocyanato-3-isocyanatomethyl-3,5,5-trimethylcyclohexane (isophorone diisocyanate or IPDI), bis-(4-isocyanatocyclohexyl)-methane, 1,3- and 1,4-bis(isocyanatomethyl)-cyclohexane, bis-(4-isocyanato-3-methyl-cyclohexyl)-methane, isomers of toluene diisocyanate (TDI) such as 2,4-diisocyanatotoluene, 2,6-diisocyanatotoluene, mixtures of these isomers, hydrogenated TDI, 4,4′-diisocyanato diphenyl methane and its isomeric mixtures with 2,4′- and optionally 2,2′-diisocyanato diphenylmethane, and 1,5-diisocyanato naphthalene. Mixtures of diisocyanates can, of course, be used. Preferred diisocyanates are aliphatic and cycloaliphatic diisocyanates. Particularly preferred are 1,6-hexamethylene diisocyanate and isophorone diisocyanate.

“Low molecular weight diols” in the context of R₃ means diols having a molecular weight from about 62 to 700, preferably 62 to 200. They may contain aliphatic, alicyclic or aromatic groups. Preferred compounds contain only aliphatic groups. The low molecular weight diols having up to about 20 carbon atoms per molecule include ethylene glycol, diethylene glycol, propane 1,2-diol, propane 1,3-diol, butane 1,4-diol, butylene 1,3-glycol, neopentyl glycol, butyl ethyl propane diol, cyclohexane diol, 1,4-cyclohexane dimethanol, hexane 1,6-diol, bisphenol A (2,2-bis(4-hydroxyphenyl)propane), hydrogenated bisphenol A (2,2-bis(4-hydroxycyclohexyl)propane), and mixtures thereof. Optionally, the low molecular weight diols may contain ionic or potentially ionic groups. Suitable lower molecular weight diols containing ionic or potentially ionic groups are those disclosed in U.S. Pat. No. 3,412,054, the contents of which is hereby incorporated by reference. Preferred compounds include dimethylol butanoic acid (DMBA), dimethylol propionic acid (DMBA) and carboxyl-containing caprolactone polyester diol. If lower molecular weight diols containing ionic or potentially ionic groups are used, they are preferably used in an amount such that <0.30 meq of COOH per gram of polyurethane in the polyurethane dispersion are present.

The prepolymer is chain extended using two classes of chain extenders. First, compounds having the formula: H₂N—R₄—NH₂ wherein R⁴ represents an alkylene or alkylene oxide radical not substituted with ionic or potentially ionic groups. Alkylene diamines include hydrazine, ethylenediamine, propylenediamine, 1,4-butylenediamine and piperazine. The alkylene oxide diamines include 3-{2-[2-(3-aminopropoxy)ethoxy]ethoxy}propylamine (also known as dipropylamine diethyleneglycol or DPA-DEG available from Tomah Products, Milton, Wis.), 2-methyl-1,5-pentanediamine (Dytec A from DuPont), hexane diamine, isophorone diamine, and 4,4-methylenedi-(cyclohexylamine), and the DPA-series ether amines available from Tomah Products, Milton, Wis., including dipropylamine propyleneglycol, dipropylamine dipropyleneglycol, dipropylamine tripropyleneglycol, dipropylamine poly(propylene glycol), dipropylamine ethyleneglycol, dipropylamine poly(ethylene glycol), dipropylamine 1,3-propane diol, dipropylamine 2-methyl-1,3-propane diol, dipropylamine 1,4-butane diol, dipropylamine 1,3-butane diol, dipropylamine 1,6-hexane diol and dipropylamine cyclohexane-1,4-dimethanol. Mixtures of the listed diamines may also be used.

The second class of chain extenders are compounds having the formula: H₂N—R₅—NH₂ wherein R₅ represents an alkylene radical substituted with ionic or potentially ionic groups. Such compounds have an ionic or potentially ionic group and two groups that are reactive with isocyanate groups. Such compounds contain two isocyanate-reactive groups and an ionic group or group capable of forming an ionic group. The ionic group or potentially ionic group can be selected from the group consisting of ternary or quaternary ammonium groups, groups convertible into such a group, a carboxyl group, a carboxylate group, a sulfonic acid group and a sulfonate group. The at least partial conversion of the groups convertible into salt groups of the type mentioned may take place before or during the mixing with water. Specific compounds include diaminosulfonates, such as for example the sodium salt of N-(2-aminoethyl)-2-aminoethane sulfonic acid (AAS) or the sodium salt of N-(2-aminoethyl)-2-aminopropionic acid.

The polyurethane according to the invention may also include compounds which are situated in each case at the chain ends and terminate said chains (chain terminators) as described in U.S. Pat. Nos. 7,445,770 and/or 7,452,770.

Preferably, the aqueous polyurethane dispersion has a viscosity of less than 2000 mPa·s at 23° C., preferably less than 1500, preferably less than 1000, including all ranges and subranges therebetween.

Also preferably, the aqueous polyurethane dispersion has a solids content based on the weight of the dispersion of from 20% to 60%, preferably from 25% to 55% and preferably from 30% to 50%, including all ranges and subranges therebetween.

According to certain embodiments, the aqueous polyurethane dispersion is made by a process in which a (polyester) copolymer or prepolymer is reacted with a (di)isocyanate, such as a non-phenylated (di)isocyanate.

Suitable aqueous polyurethane dispersions for use in the present invention include, but are not limited to, aqueous polyurethane dispersions sold under the BAYCUSAN® name by Covestro such as, for example, BAYCUSAN® C1001 (polyurethane-34), BAYCUSAN® C1003 (polyurethane-32), and BAYCUSAN® C1004 (polyurethane-35).

Polyurethane-34 is made by a process where a copolymer of Hexanediol (q.v.), Neopentyl Glycol (q.v.), and Adipic Acid (q.v.) is reacted with hexamethylene diisocyanate (a non-phenylated diisocyanate). The resulting polymer is further reacted with sodium N-(2-aminoethyl)-2-aminoethane sulfonate and ethylenediamine.

Polyurethane-35 is made by a process that includes reacting a polyester pre-polymer consisting of Adipic Acid (q.v.), Hexanediol (q.v.), and Neopentyl Glycol (q.v.) with Saturated Methylene Diphenyl Diisocyanate (q.v.). The resulting urethane polymer is further reacted with sodium N-(2-aminoethyl)-2-aminoethanesulfonate and ethylenediamine and then dispersed into water.

In more preferred embodiments, the aqueous polyurethane dispersion includes polyurethane-34 and/or polyurethane-35. In a more preferred embodiment, the aqueous polyurethane dispersion includes or consists of polyurethane-34.

In some embodiments, the aqueous polyurethane dispersions may be present in amounts from 2%, 4%, 5%, 8% or 10% to about 10%, 12%, 15%, 20%, or 25% by weight of the composition, including any combinations or subrange thereof.

According to certain embodiments, the one or more acrylic latex polymers having a low glass transition temperature and the one or more aqueous polyurethane dispersions are present in a ratio of concentration by weight of one or more acrylic latex polymers having a low glass transition temperature to concentration by weight of one or more aqueous polyurethane dispersions that is in a range from about 1:3, 1:2.5, 1:2, 1:1.5, or 1:1, to about 1:1, 1.5:1, 2:1, 2.5:1 or 3:1. This ratio may most desirably be between about 1:1 and about 3:1, such as between about 1:1 to about 2:1.

### Saccharide Ester of at Least One C2-C4 Organic Acid

Cosmetic adhesive compositions of the present invention include a saccharide ester of at least one C2-C4 organic acid. The saccharide ester of at least one C2-C4 organic acid may be useful to accelerate the onset of tack. Examples of suitable saccharide esters include those soluble in C2-C4 monoalcohols and/or ethyl acetate. According to certain embodiments, the C2-C4 organic acid is or includes acetic acid and butyric acid. According to certain embodiments the cellulose ester of at least one C2-C4 organic acid is or includes sucrose acetate isobutyrate (“SAIB”).

The saccharide ester of at least one C2-C4 organic acid may be, for example, sucrose acetate butyrate commercially available from Eastman Chemical such as SAIB-100.

The cellulose ester of at least one C2-C4 organic acid may be present in a concentration by weight in the nail topcoat composition that is from about 0.5%, 1%, 2% 3% or 5% to about 8%, 10%, 15, 20%, 25% or 30%.

Additional adhesive polymers other than the one or more acrylic latex polymers having a low glass transition temperature, the one or more aqueous polyurethane dispersions, and the one or more saccharide ester of at least one C2-C4 organic acid may be present in the adhesive cosmetic composition. Examples of other adhesive polymers include acrylate polymers having a T_{g} no more than about 20° C.; polysaccharides including various gums such as xanthan gum, gellan gum, and the like; starches and starch derivatives such as sodium polyacrylate starch, cellulose and its derivatives; certain vinyl polymers such as polyvinyl acetate, and the like. In certain embodiments, the additional adhesive polymer when tested according to the Initial Adhesive Test or Dry Adhesion Test, both described herein have values of at least 2, such as at least 3.

In certain embodiments, the additional adhesive polymers are present in a total concentration by weight that is less than the total concentration by weight of the one or more acrylic latex polymers having a low glass transition temperature, the one or more aqueous polyurethane dispersions, and the one or more saccharide ester of at least one C2-C4 organic acid. In certain embodiments, the additional adhesive polymers are present in a total concentration by weight that is less than any individual concentration by weight of the one or more acrylic latex polymers having a low glass transition temperature, the one or more aqueous polyurethane dispersions, and the one or more saccharide ester of at least one C2-C4 organic acid. In certain embodiments the cosmetic adhesive composition is substantially free of additional adhesive polymers.

Certain classes of compounds may be optionally sufficiently excluded from the cosmetic adhesive compositions of the present invention—so as to be substantially free or free of such compound(s). For example, the cosmetic adhesive composition may be free or substantially free one, a plurality or all of: waxes, oils, silicones and C2-C6 polyols.

### Waxes

By waxes, it is meant a lipophilic compound that is solid at room temperature (25° C.), with a solid/liquid reversible change of state, having a melting point of greater than or equal to 30° C., which may be up to 200° C. and in particular up to 120° C. Waxes may be soft or hard.

As used herein, the term “soft wax” refers to waxes which have a melting point of below about 70° C., and preferably, a melting point of below about 60° C. As used herein, the term “hard wax” refers to waxes other than soft waxes—that is, waxes which have a melting point of equal to or greater than about 70° C., and preferably, a melting point of equal to or greater than about 60° C.

Non-limiting examples of soft waxes include paraffin wax, ozokerite, synthetic beeswax, beeswax, candelilla wax, hydrogenated jojoba wax, palm butter, sumac wax, polyglyceryl beeswax, siliconyl beeswax, ceresin wax, orange peel wax, silicone resin wax, alkyl dimethicone wax, Berry wax, koster wax, siliconyl candelilla wax, and montan wax.

Non-limiting examples of hard waxes include carnauba wax, microcrystalline wax, polyethylene wax, hydrogenated castor oil, wax AC 540, hydrogenated castor wax, wax AC 400, rice bran wax, Alcohol polyethylene wax, sunflower seed wax, fischer-tropsch wax, Chinese insect wax, and shellac wax.

### Oils

“Oil” means any non-aqueous medium which is liquid at ambient temperature (25° C.) and atmospheric pressure (760 mm Hg). Oils are generally insoluble in water and include a hydrophobic moiety, such as one meeting one or more of the following three criteria: (a) has a (n uninterrupted) carbon chain of at least six carbons, such as at least seven carbons, such as at least eight carbons, in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. The hydrophobic compound is in certain embodiments not amphiphilic and, as such, in this embodiment does not include hydrophilic moieties, such as anionic, cationic, zwitterionic, or nonionic groups, that are polar, including sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy) sulfonyl moieties. In certain embodiments, the oil does not include hydroxyl moieties. According to certain other embodiments, oils have a molecular weight less than about 800 daltons.

Suitable examples of compounds of oils include vegetable oils (glyceryl esters of fatty acids, monoglycerides, diglycerides, triglycerides) and fatty esters. Specific non-limiting examples include, without limitation, esters such as isopropyl palmitate, isopropyl myristate, isononyl isonanoate C₁₂-C₁₅ alkyl benzoates, caprylic/capric triglycerides, ethylhexyl hydroxystearate, silicone oils (such as dimethicone and cyclopentasiloxane), pentaerythritol tetraoctanoate and mineral oil. Other examples of oils include liquid organic ultraviolet filter commonly used for example as UV-absorbing sunscreens such as octocrylene, octyl salicylate, octyl methoxyxcinnamate, among others.

Suitable oils include volatile and/or non-volatile oils. Such oils can be any acceptable oil including but not limited to silicone oils and/or hydrocarbon oils. Examples of volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures.

C2-C6 Polyols

While in certain embodiments C2-C6 polyols are included in compositions of the present invention, in some embodiments, the composition may be substantially free of C2-C6 polyols. Non-limiting examples of C2-C6 polyols include glycerol or diols, such as, 1,2-pentanediol, propanediol, butanediol, glycols and glycol ethers, such as propylene glycol, butylene glycol, dipropylene glycol or diethylene glycol.

### Silicones

In some embodiments, the composition may be substantially free of silicones. By silicones, it is meant materials having a plurality of Si—O—R units where R can be any organic group.

### Other Materials

In some embodiments, the composition may comprise other materials. The other materials may be present in a total amount of less than 40% and in certain embodiments, less than about 20% by weight of the composition. In some embodiments, the other materials may be present in a total amount of 25-35% by weight of the composition.

In some embodiments, the other materials may comprise one or more cosmetic ingredients selected from colorants, C2-C8 monoalcohols, C2-C6 polyols, chelating agents, antioxidants, preservatives, and surfactants. Such other materials may collectively be present in a concentration by weight from about 0%, 1% 2% 3% or 5% to about 5%, 10% 15% or 20%. In certain embodiments, the compositions include C2-C4 monoalcohols in concentrations ranging from about 0%, 0.5%, or 1% to about 1%, 2%, 3%, 5%, or 10%.

For purposes of this disclosure, the one or more acrylic latex polymers having a glass transition temperature (T_{g}) less than about −40° C.; the one or more aqueous polyurethane dispersions; and the one or more saccharide esters of at least one C2-C4 organic acid—are excluded from the definition of “other materials,” even if these adhesive polymers would otherwise meet the functional definition of one or more of these classes of ingredients.

### Colorants

The compositions of the present invention may optionally further comprise at least one colorant. Suitable colorants (coloring agents) include any colorant typically found in cosmetic compositions. Suitable colorants include, but are not limited to, lipophilic dyes, pigments and pearlescent agents, and their mixtures.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

The colorant may be present in an amount sufficient to provide color to lashes. In some embodiments, the colorant may be present in a total amount from 0.1% to 20% by weight of the composition. In some embodiments, the colorant may be present in a total amount from 0%, 0.1%, 0.5%, 1%, 2%, 3%, 4%, or 5%, up to 10%, 12.5%, or 15% by weight of the composition, including any combination or subrange thereof.

### C2-C8 Monoalcohols

In some embodiments, the composition may include one or more C2-C8 monoalcohols, such as C3-C4 monoalcohols. In some embodiments, a single C2-C8 monoalcohol is utilized. In some embodiments, the C2-C8 monoalcohol is a C2-C8 monoalkanol.

Non-limiting examples of C2-C8 monoalcohols include ethanol and isopropanol. If the C2-C8 monoalcohol is ethanol, the C2-C8 monoalcohol includes any denaturant used to denature the ethanol. In some embodiments, the C2-C8 monoalcohol is a denatured alcohol.

In some embodiments, the C2-C8 monoalcohol may be present in a total amount of 0.1%-5% by weight of the composition. In some embodiments, the C2-C8 monoalcohol may be present in a total amount from 0.1%, 1.5%, 2%, or 3% up to 4%, 4.5%, or 5% by weight of the composition, including any combination or subrange thereof.

### Thickening Agent

In some embodiments, the composition may include a thickening agent. In some embodiments, the composition includes a single thickening agent. In some embodiments, the composition includes a plurality of thickening agents.

In some embodiments, the thickening agent may be a polymer of natural origin. Non-limiting examples of polymers of natural origin useful as a thickening agent include thickening polymers comprising at least one sugar unit, for instance nonionic guar gums, optionally modified with C1-C6 hydroxyalkyl groups; biopolysaccharide gums of microbial origin, such as scleroglucan gum or xanthan gum; gums derived from plant exudates, such as gum arabic, ghatti gum, karaya gum, gum tragacanth, carrageenan gum, agar gum and carob gum; pectins; alginates; starches; hydroxy(C1-C6)alkylcelluloses and carboxy(C1-C6)alkylcelluloses.

In some embodiments, the thickening agent may be a cellulosic thickener. Non-limiting examples of cellulose thickeners include hydroxyethylcellulose, hydroxymethylcellulose, hydroxypropylcellulose, methylcellulose, ethylhydroxyethylcellulose, carboxymethylcellulose, starches such as sodium polyacrylate starch, and mixtures thereof.

In some embodiments, the thickening agent may be present in a total amount of 0%-10% by weight of the composition. In some embodiments, the thickening agent may be present in a total amount from 0.1%, 0.25%, or 0.5% up to 1%, 2%, 3%, 4%, 5%, 6%, 7%, 8%, 9%, or 10% by weight of the composition, including any combination or subrange thereof. In some embodiments, the thickening agent may be present in a total amount of 0.1%-2% by weight of the composition. In some embodiments, the composition may be free or substantially free of a thickening agent.

### Surfactants/Emulsifiers

In some embodiments, the compositions may include one or more surfactants. In some embodiments, the compositions may include a plurality of surfactants. As one skilled in the art will recognize, surfactants and emulsifiers are generally amphiphilic molecules and capable of depressing surface tension of deionized water to levels less than about 30 dynes/cm.

In some embodiments, the composition is free or substantially free of cationic surfactants or free of surfactants entirely.

In some embodiments, the composition may include one or more anionic surfactants. Examples include alkali metal or other salts such as sodium salts, ammonium salts, amine salts, amino alcohol salts and alkaline-earth metal salts, for example magnesium salts, of the following types of compounds: alkyl sulfates, alkyl ether sulfates, acyl isethionates, acyl glycianates, acyl taurates, acyl amino acids, acyl sarcosinates, sulfosuccinates, sulfonates, the alkyl and acyl groups of all these compounds comprising from 6 to 24 carbon atoms (saturated or unsaturated, linear or branched).

In some embodiments, the composition may include one or more or one or more nonionic surfactants. Non-limiting examples of nonionic surfactants include alkoxylated derivatives of the following: fatty alcohols, alkyl phenols, fatty acids, fatty acid esters such as sorbitan fatty acid esters, and fatty acid amides, wherein the alkyl chain is in the C12-C50 range, preferably in the C16-C40 range, more preferably in the C24 to C40 range, and having from about 1 to about 110 alkoxy groups. The alkoxy groups may be selected from the group consisting of C2-C6 oxides and their mixtures, with ethylene oxide, propylene oxide, and their mixtures being the preferred alkoxides. The alkyl chain may be linear, branched, saturated, or unsaturated. The alkoxylated alcohols may be used alone or in mixtures thereof.

### Antioxidants, Chelating Agents, and Preservatives

In some embodiments, the composition may include an antioxidant, chelating agent, and/or preservative. In some embodiments, the composition may be substantially free of an antioxidant, a chelating agent, and/or a preservative. The optional antioxidant, a chelating agent, and/or a preservatives may be selected from commonly used cosmetic examples of these functional materials.

In some embodiments, a method may be provided. The method includes applying a compositions described herein to natural lashes or bows and/or to false lashes (strip, cluster, etc.), false brows or fibers to be adhered to natural lashes or a natural brow area. Used herein the term “lash/brow” refers to either or both lash or brow. The false lashes, brows, or fibers may then be positioned so as to form an augmented lash or augmented brow structure.

According to certain aspects, a method of adhering a false eyelash or brow includes (1) applying the composition described above to a surface selected from a lash or brow to form an active adhesive surface. It further includes (2) contacting the active adhesive surface with a corresponding mating surface of a lash or brow to form an augmented lash or brow on the user. For example, the adhesive composition may be applied to a false lash and then the active adhesive surface of the false lash may be attached to a corresponding natural lash to form an augmented lash. Similarly, the adhesive may be applied to false brow, creating an active adhesive surface and then this is attached to a corresponding natural brow to form an augmented brow. Alternatively, the adhesive may be applied to a natural lash forming an active adhesive surface thereon and then this is contacted with a corresponding false lash to attached it thereto. Similarly, the adhesive may be applied to a natural brow and then a corresponding false brow (or fibers) may be attached to the natural brow. In other embodiments, the cosmetic adhesive composition is applied to a skin surface such as skin on the eyelid or skin of the brow area.

### EXAMPLES

### Example 1

Three raw materials, a acrylic latex polymer having a glass transition temperature (T_{g}) less than about −40° C. (Acrylates/Ethylhexyl Acrylate Copolymer, 55% active in water), an aqueous polyurethane dispersion (Polyurethane-34, 32% active in water) and a saccharide esters of at least one C2-C4 organic acid (Sucrose Acetate Isobutyrate, 90% active in alcohol) were separately evaluated for the following characteristics: initial adhesion, dry adhesion, and cohesion-which the inventors have found to be characteristic of high performing all day lash glue: (1) Initial (wet) adhesion, (2) dry adhesion, and (3) cohesion. These tests are described below.

### Evaluation: Initial (Wet) Adhesion

A droplet of each composition was deposited on dry skin and spread to uniformity. The deposit was monitored for sufficient dryness to evaluate tack. As the deposit transitioned to clear (i.e., at the start of the drying period of the deposit) the tackiness was rated qualitatively from 1 to 5 (1=weak/low tack, 5=highly tacky), by pressing a finger to the deposit and pulling finger in an upward direction, and gauging the force needed to separate fingers. Ratings are assigned as per the following: a (1) was defined as a composition with extremely low initial tack (little to no adhesion to finger at any timepoint while drying/freshly dry)—an example is Polyurethane-6, available as LUVISET Si-P.U.R.A, available from BASF. A (3) was defined as a film with intermediate resistance to peeling from weigh boat; an example is commercial glue Ardell Brush-On Eyelash Adhesive (ingredients: Alcohol, water, Acrylate Copolymer, Sorbitol). A (5) was defined as a composition with very high initial tack (high adhesion to finger, to the point of difficulty of removing finger from film). an example is Acrylates/ethylhexyl acrylate copolymer, Daitosol 5500GX.

### Evaluation: Dry Adhesion

Approximately 7 g of each composition was poured in a polystyrene weigh boat and allowed to dry overnight in ambient conditions or in a fume hood. The adhesion of the dried composition to the weigh boat was rated qualitatively from 1 to 5 (1=low adhesion, 5=high adhesion) by pulling film away from weigh boat, gauging the force needed to remove film from weigh boat surface. Ratings are assigned as per the following: a (1) was defined as a film that could be removed from weigh boat with little to no force, an example is sucrose acetate isobutyrate; a (5) was defined as a film that was very difficult to remove from the weigh boat. An example is Polyurethane-34.

### Evaluation: Cohesion

Approximately 7 g of each composition was poured in a weigh boat and allowed to dry overnight in ambient conditions or in a fume hood. The cohesion of the dried composition was rated qualitatively from 1 to 5 (1=low cohesion, 5=high cohesion) by removing a segment of film from the weigh boat, and gauging the force needed to create a rupture or tear in that segment. Ratings are assigned as per the following: a (1) was defined as a film that could be very easily ripped apart. An example is sucrose acetate isobutyrate; a (5) was defined as a film that was extremely difficult to rip apart, an example is Polyurethane-34.

Evaluation: Dry Time [can we Say Anything about how Dry Time was Determined?]

Dry time was determined by monitoring film on skin for visual transition from white to clear.

The test results for Example 1 are shown below.

TABLE 1
Comparative Formulas (Raw Materials)

| Ingredients | C1 | C2 | C3 |
|---|---|---|---|
| Acrylates/Ethylhexyl | 55% | | |
| Acrylate Copolymer | | | |
| Polyurethane-34 | | 32% | |
| Sucrose Acetate | | | 90% |
| Isobutyrate | | | |
| Initial Adhesion | 5 | 2 | 4 |
| Dry Adhesion | 4 | 5 | 1 |
| Cohesion | 2 | 5 | 1 |

The results show that none of the three components separately possess sufficient performance in all three properties.

### Example 2

The following formulas were produced, by one of the following processes: (A), (B) or (C). (A) Combining all components described below by speed mixing using a Hauschild SpeedMixer® at 2000-2500 RPM for up to 5 minutes, at room temperature, (B) Mixing all components together on a Rayneri mixer at room temperature using a Cowles blade, or (C) A close variant of process ‘B,’ but adapted for compositions without alcohol as a co-solvent, for improved dispersion of sucrose acetate isobutyrate. (C) Included combining oil-soluble components in a first container, heating and mixing until homogenous; combining remaining components in a second container, heating and mixing until homogenous; combining containers together, homogenizing, cooling, and pouring into an appropriate product container. The particular process used for each composition, as well as the ingredients by weight percentage, are noted in Table 2, below.

TABLE 2
Comparative Formulas (Sucrose Acetate Isobutyrate + Polyurethane-34)

| | C4 | C5 | C6 | C7 | C8 | C9 |
|---|---|---|---|---|---|---|
| Ingredients | (Fla 1P) | (Fla 2P) | (Fla 3P) | (Fla 4P) | (Fla 5P) | (Fla 6P) |
| Sucrose Acetate | 5% | 5% | 10% | 20% | 30% | 50% |
| Isobutyrate | | | | | | |
| (SAIB) | | | | | | |
| Polyurethane- | 30.22% | 20% | 10% | 5% | 5% | 5% |
| 34 | | | | | | |
| Water | 62.81% | 73.50% | 78.42% | 72.55% | 61.44% | 39.22% |
| Emulsifiers | 0.56% | 0.56% | 1.11% | 2.22% | 3.33% | 5.56% |
| Preservatives | 1.41% | 0.94% | 0.47% | 0.23% | 0.23% | 0.23% |
| Process used | C | C | C | C | C | C |
| Initial Adhesion | 1.5 | 1.5 | 3 | 3 | 3 | 3 |
| Dry Adhesion | 3 | 2 | 2 | 2 | 2.5 | 1 |
| Cohesion | 4.5 | 4 | 4 | 2 | 2.5 | 1.5 |
| Dry Time (s) | 90-100 | 90-100 | 80-90 | 80-90 | 60-80 | 60-80 |

The results in Table 2 show that when the aqueous polyurethane dispersion and saccharide esters of at least one C2-C4 organic acid are used without the acrylic latex polymer having a glass transition temperature (T_{g}) less than about −40° C., at least initial adhesion and dry adhesion are unacceptably low. In some cases (Comparative Examples, C4 and C5), initial adhesion for the blend is even worse than the poor initial adhesion of the polyurethane dispersion alone (see Example 1). It was also found that C4, C5, C6, and C7 had stability problems. Surprisingly, however, it was found that SAIB in higher concentrations accelerated the onset of tack/adhesion.

### Example 3

The following formulas were produced and tested.

TABLE 3
Comparative Formulas (Polyurethane-34 +
Acrylates/Ethylhexyl Acrylate Copolymer)

| | | | | C13 | C13 |
|---|---|---|---|---|---|
| | C10 | C11 | C12 | (Fla | (Fla |
| Ingredients | (Fla 13P) | (Fla 15P) | (Fla 16P) | 19P) | 14P) |
| Acrylates/ | 3.03% | 5% | 20.35% | 30.25% | 46.4% |
| Ethylhexyl | | | | | |
| Acrylate | | | | | |
| Copolymer | | | | | |
| Polyurethane- | 27.2% | 20% | 20.16% | 7.36% | 5% |
| 34 | | | | | |
| Water | 68.47% | 74.01% | 58.36% | 61.77% | 47.94% |
| Preservatives | 1.3% | 0.99% | 1.13% | 0.62% | 0.66% |
| Process used | A | A | A | A | A |
| Initial | 2 | 2.5 | 5 | 4 | 4.5 |
| Adhesion | | | | | |
| Dry Adhesion | 4.5 | 4.5 | 5 | 4 | 2 |
| Cohesion | 4 | 4 | 5 | 3 | 1.5 |
| Dry Time (s) | 90-120 | 90-120 | 90-120 | 90-120 | 90-120 |

The results in Table 3 show that when the acrylic latex polymer having a glass transition temperature (T_{g}) less than about −40° C. and the aqueous polyurethane dispersion are used without the saccharide esters of at least one C2-C4 organic acid, it is possible to achieve a good balance of properties (see C12) if the concentrations of acrylic latex polymer having a glass transition temperature (T_{g}) less than about −40° C. and aqueous polyurethane dispersion are not too different from one another. However, it was observed that C12 had an unacceptably long dry time (greater than 90 seconds).

### Example 4

The following formulas were produced and tested.

TABLE 4
Comparative Formulas (Acrylates/Ethylhexyl Acrylate
Copolymer + Sucrose Acetate Isobutyrate)

| | C 14 | C 15 | C 16 |
|---|---|---|---|
| Ingredients | (Fla 7P) | (Fla 28P) | (Fla 11P) |
| Acrylates/ Ethylhexyl | 50% | 45.82% | 4.95% |
| Acrylate Copolymer | | | |
| Sucrose Acetate | 5% | 15.03% | 50% |
| Isobutyrate | | | |
| Water | 44% | 37.07% | 39.46% |
| Emulsifiers | 0.56% | — | 5.56% |
| Alcohol | — | 1.67% | — |
| Preservatives | 0.44% | 0.41% | 0.03% |
| Process used | C | A | C |
| Initial Adhesion | 3.5 | 3 | 2 |
| Dry Adhesion | 5 | 3 | 1.5 |
| Cohesion | 1.5 | 3 | 2 |
| Dry Time (s) | 80-90 | 80-90 | 80-90 |

The results in Table 4 show that when the acrylic latex polymer having a glass transition temperature (T_{g}) less than about −40° C. and the saccharide esters of at least one C2-C4 organic acid are used without the aqueous polyurethane dispersion, the initial adhesion is worse than the initial adhesion for either of the raw materials alone (see Example 1).

### Example 5

The following formulas using all three of acrylic latex polymer having a glass transition temperature (T_{g}) less than about −40° C., aqueous polyurethane dispersion, and saccharide esters of at least one C2-C4 organic acid were prepared and tested.

TABLE 5
(Inventive and Comparative Formulas)

| | Ex 1 | Ex 2 | Ex 3 | C 1 | C 2 |
|---|---|---|---|---|---|
| | (Fla | (Fla | (Fla | (Fla | (Fla |
| Ingredients | 34) | 11B) | 17P) | 18P) | 11G) |
| Sucrose | 30% | 6.8% | 30% | 13.5% | 6.80% |
| Acetate | | | | | |
| Isobutyrate | | | | | |
| Polyurethane- | 10.7% | 18.6% | 10% | 13.36% | 14.58% |
| 34 | | | | | |
| Acrylates/ | 18.3% | 18.5% | 10% | 22.96% | 25.05% |
| Ethylhexyl | | | | | |
| Acrylate | | | | | |
| Copolymer | | | | | |
| Water | 36.96% | 53.56% | 46.1% | 46.34% | 50.56% |
| Emulsifiers | — | 0.75% | 3.3% | 1.5% | 0.75% |
| Alcohol | 3.34% | 0.75% | — | 1.5% | 0.755% |
| Preservatives | 0.7% | 0.97% | 0.6% | 0.84% | 1.5% |
| Thickener | — | 0.07% | — | — | — |
| Process used | A | B | C | A | A |
| Initial | 5 | 4 | 4 | 4 | 4 |
| Adhesion | | | | | |
| Dry Adhesion | 5 | 4 | 4 | 3 | 5 |
| Cohesion | 5 | 4 | 4 | 3 | 2.5 |
| Dry Time (s) | 60-80 | 60-80 | 80-90 | 80-90 | 60-80 |

The results in Table 5 show that when all three of acrylic latex polymer having a glass transition temperature (T_{g}) less than about −40° C., aqueous polyurethane dispersion, and saccharide esters of at least one C2-C4 organic acid are included and the ratio of concentrations by weight of acrylic latex polymer having a glass transition temperature (T_{g}) less than about −40° C. to aqueous polyurethane dispersion is from 1:3 to 3:1, and from about 10% to 20% by weight of acrylic latex polymer having a glass transition temperature (T_{g}) less than about −40° C., possessed a balance of adhesive/cohesive properties that is surprisingly very good with acceptable dry time.

Those skilled in the art will recognize or be able to ascertain using no more than routine experimentation many equivalents to the specific embodiments of the invention described herein. Such equivalents are intended to be encompassed by the following claims.

What is claimed:

1. A cosmetic adhesive composition, comprising:
water;
from about 10% to 20% by weight of one or more acrylic latex polymers having a glass transition temperature (T_{g}) less than about −40° C.;
one or more aqueous polyurethane dispersions;
one or more saccharide esters of at least one C2-C4 organic acid; wherein the one or more acrylic latex polymers having a low glass transition temperature and the one or more aqueous polyurethane dispersions are present in a ratio of concentration by weight of the one or more acrylic latex polymers having a low glass transition temperature to concentration by weight of the one or more aqueous polyurethane dispersions that is in a range from about 1:3 to about 3:1.

2. The composition according to claim 1, wherein the one or more acrylic latex polymers having a low glass transition temperature and the one or more aqueous polyurethane dispersions are present in a ratio of concentration by weight of the one or more acrylic latex polymers having a low glass transition temperature to concentration by weight of the one or more aqueous polyurethane dispersions that is in a range from about 1:1 to about 2:1.

3. The composition according to claim 1, wherein the one or more acrylic latex polymers having a glass transition temperature (T_{g}) less than about −40° C. has a T_{g} less than about −50° C.

4. The composition according to claim 1, wherein the one or more aqueous polyurethane dispersions is formed by reacting a polyester prepolymer with a non-phenylated isocyanate.

5. The composition according to claim 1, wherein the composition is substantially free of one or more of waxes, oils, silicones and/or C2-C6 polyols.

6. The composition according to claim 1, wherein the composition is substantially free of each of waxes, oils, silicones and C2-C6 polyols.

7. The composition according to claim 1, wherein the composition optionally includes one or more additional adhesive polymers, and wherein the total concentration by weight of the one or more acrylic latex polymers having a glass transition temperature (T_{g}) less than about −40° C.; the one or more aqueous polyurethane dispersions; the one or more saccharide esters of at least one C2-C4 organic acid, and the one or more additional adhesive polymers is in a range from about 30% to about 70%.

8. The composition according to claim 1, wherein the composition optionally includes one or more additional adhesive polymers, and wherein the total concentration by weight of the one or more acrylic latex polymers having a glass transition temperature (T_{g}) less than about −40° C.; the one or more aqueous polyurethane dispersions; the one or more saccharide esters of at least one C2-C4 organic acid, and the one or more additional adhesive polymers is in a range from about 40% to about 60%.

9. The composition according to claim 1, wherein one or more aqueous polyurethane dispersions is present in the composition in a concentration by weight from about 8% to about 20% of the composition.

10. The composition according to claim 1, wherein one or more saccharide esters of at least one C2-C4 organic acid is present in an amount of 5% to about 30% by weight of the composition.

11. The composition of claim 1, wherein the composition is substantially free of additional adhesive polymers.

12. A method of adhering a false eyelash/brow to a corresponding natural lash/brow, comprising
applying the composition of claim 1 to a surface selected from the false lash/brow, the corresponding natural lash/brow, a skin surface or combinations thereof to form at least one active adhesive surface; and
contacting the at least one active adhesive surface with a corresponding mating surface to form an augmented lash/brow.
